(19) United States
(12) Patent Application Publication
(10) Pub. No.: US 2026/0248530 A1
(43) Pub. Date: Aug. 27, 2026

(54) TISSUE LACERATION DEVICE

(71) Applicant: BOSTON SCIENTIFIC SCIMED, INC., MAPLE GROVE, MN (US)

(72) Inventors: James M. Anderson, Corcoran, MN (US); Eric Michael Petersen, Maple Grove, MN (US); Lauren Koon, Etobicoke (CA); Phil Litecky, Forest Lake, MN (US)

(73) Assignee: BOSTON SCIENTIFIC SCIMED, INC., MAPLE GROVE, MN (US)

(21) Appl. No.: 19/548,139

(22) Filed: Feb. 24, 2026

Related U.S. Application Data
(60) Provisional application No. 63/762,292, filed on Feb. 24, 2025.

(51) Int. Cl. A61B 17/3209; A61B 17/32

(52) U.S. Cl. CPC A61B 17/3209; A61B 2017/320048

(57) ABSTRACT

A medical device for lacerating tissue includes an elongate shaft with a distal region and an inflatable balloon secured to the distal region. The balloon has an outer surface and can transition between deflated and inflated configurations. The device features an electrode assembly on the outer surface with multiple electrodes optionally arranged in a grid pattern. The balloon may be constructed of polyimide or polyamide materials, potentially with multiple layers. The electrodes can be individually activated to create various cutting patterns and may be arranged axially or non-parallel to each other. Some embodiments include an insulative surface adhered to the balloon's outer surface with a conductive pattern defining the electrodes. The electrode assembly may be radiopaque and can include monopolar annular electrodes with separate ground electrodes.

### CROSS REFERENCE TO RELATED APPLICATIONS

This application claims the benefit of priority of U.S. Provisional Application No. 63/762,292 filed February 24, 2025, the entire disclosure of which is hereby incorporated by reference.

### TECHNICAL FIELD

The present disclosure relates generally to medical devices. More particularly, the present disclosure pertains to medical devices for lacerating tissue such as cardiac valve leaflets.

### BACKGROUND

A wide variety of intracorporeal medical devices have been developed for medical use, and more specifically for intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for lacerating tissue such as cardiac valve leaflets. An example may be found in a medical device that is adapted for lacerating valve leaflets. The medical device includes an elongate shaft that extends proximally from a distal region and an inflatable balloon that is secured to the distal region. The inflatable balloon has an outer surface and includes a deflated configuration and an inflated configuration. An electrode assembly is disposed relative to the outer surface and includes a plurality of electrodes.

Alternatively or additionally, the inflatable balloon may include a polyimide polymer or a polyamide polymer.

Alternatively or additionally, the inflatable balloon may include an inner layer including a polyamide polymer and an outer layer including a polyimide polymer.

Alternatively or additionally, the plurality of electrodes may be arranged in an x by y grid, where x and y are each integers that are equal to or greater than one.

Alternatively or additionally, x may range from one to ten and y may range from one to ten.

Alternatively or additionally, each of the plurality of electrodes may be electrically isolated from each other.

Alternatively or additionally, each of the plurality of electrodes may be individually actuatable by electrically coupling a source of RF energy with individual electrodes of the plurality of electrodes.

Alternatively or additionally, the electrode assembly may be adapted to allow varying lacerating or cauterizing patterns by individually actuating individual electrodes of the plurality of electrodes.

Alternatively or additionally, the electrode assembly may include two or more axially aligned electrodes.

Alternatively or additionally, the electrode assembly may include two or more elongate electrodes that are not parallel to each other.

Alternatively or additionally, the plurality of electrodes may include one or more monopolar electrodes.

Another example may be found in a medical device is adapted for lacerating cardiac tissue. The medical device includes an elongate shaft that extends proximally from a distal region and an inflatable balloon that is secured to the distal region. The inflatable balloon has an outer surface and includes a deflated configuration and an inflated configuration. An electrode assembly is disposed on the outer surface. The electrode assembly includes an insulative surface that is adhered to the outer surface and a conductive pattern that is formed on the insulative surface. The conductive pattern may define a plurality of electrodes.

Alternatively or additionally, the inflatable balloon may include a polyimide polymer or a polyamide polymer.

Alternatively or additionally, each of the plurality of electrodes may be electrically isolated from each other and individually actuatable.

Alternatively or additionally, the electrode assembly may be adapted to allow varying cutting patterns by individually actuating individual electrodes of the plurality of electrodes.

Alternatively or additionally, the plurality of electrodes may include two or more axially aligned electrodes.

Alternatively or additionally, the plurality of electrodes may include two or more non-parallel elongate electrodes.

Alternatively or additionally, the electrode assembly may be radiopaque.

Another example may be found in a medical device that is adapted for lacerating or cauterizing valve leaflets. The medical device includes an elongate shaft that extends proximally from a distal region and an inflatable balloon that is secured to the distal region. The inflatable balloon includes a polyimide or a polyamide and has an outer surface. An electrode assembly is disposed relative to the outer surface. The electrode assembly includes a plurality of electrodes that are arranged in an x by y grid, where x and y are each integers that are equal to or greater than one.

Alternatively or additionally, the electrode assembly may be adapted to allow varying cutting patterns by individually actuating individual electrodes of the plurality of electrodes.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing an aortic heart valve positioned within a native valve annulus of a heart;

FIG. 2 is a schematic view of an illustrative medical device shown in FIG. 1;

FIG. 3 is a cross-sectional view taken along the line 3-3 of FIG. 2;

FIG. 4 is a schematic view of an illustrative electrode assembly forming part of the illustrative medical device of FIG. 2;

FIG. 5 is a schematic view of an illustrative electrode assembly forming part of the illustrative medical device of FIG. 2;

FIG. 6 is a schematic view of an illustrative electrode assembly forming part of the illustrative medical device of FIG. 2, showing electrode actuation for a particular cutting pattern;

FIG. 7 is a schematic view of an illustrative electrode assembly forming part of the illustrative medical device of FIG. 2, showing electrode actuation for a particular cutting pattern;

FIG. 8 is a schematic view of an illustrative electrode assembly forming part of the illustrative medical device of FIG. 2, showing electrode actuation for a particular cutting pattern;

FIG. 9 is a schematic view of an illustrative electrode assembly forming part of the illustrative medical device of FIG. 2, showing electrode actuation for a particular cutting pattern; and

FIG. 10 is a schematic view of a portion of an illustrative electrode assembly showing that the individual electrodes are individually addressable and actuatable.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

The medical devices described herein may be used for lacerating tissue in a variety of different locations within the body, including various locations within the heart. For example, the medical devices described herein may be used for lacerating aortic valve leaflets, mitral valve leaflets and tricuspid valve leaflets. In some cases, the medical devices described herein may be used for lacerating native valve leaflet material. In some cases, the medical devices described herein may be used for lacerating artificial or replacement valve leaflet material within a replacement cardiac valve.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove or excise one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine pericardium, or may be polymeric. In some instances, artificial valve leaflets may be made of polymers such as Dacron or Gore-Tex. As discussed here, reference to a valve leaflet may refer to either a native valve leaflet or an artificial valve leaflet.

As noted, the medical devices described herein may be used in excising portions of valve leaflets regardless of whether the valve leaflets are native valve leaflets or artificial valve leaflets. In some cases, portions of the native valve leaflets may be excised prior to implantation of an artificial heart valve in order to avoid possible issues with one or more of the native valve leaflets from obscuring an ostium of one of the coronary arteries. Even if blood is able to flow through the ostium and into one of the coronary arteries, having the ostium even partially blocked with a native valve leaflet can potentially cause difficulties with subsequent procedures such as performing angioplasty within one of the coronary arteries or implanting a stent within one of the coronary arteries.

In some cases, a second artificial heart valve may be implanted within a previously implanted artificial heart valve. There may be a desire to excise one or more of the artificial valve leaflets within the previously implanted artificial heart valve before implanting the replacement artificial heart valve within the previously implanted artificial heart valve. In some cases, excising one or more of the artificial valve leaflets may help reduce or eliminate potential issues with the artificial valve leaflets of the previously implanted artificial heart valve interfering with operation of the replacement artificial heart valve and/or potentially blocking an ostium of one of the coronary arteries.

In some instances, a medical device is adapted for lacerating valve leaflets. The medical device includes an elongate shaft that extends proximally from a distal region and an inflatable balloon that is secured to the distal region. The inflatable balloon has an outer surface and includes a deflated configuration and an inflated configuration. An electrode assembly is disposed relative to the outer surface. The electrode assembly includes a plurality of electrodes. In some cases, the inflatable balloon includes a polyimide polymer or a polyamide polymer. In some cases, the inflatable balloon may include an inner layer and an outer layer. In some cases, the inner layer may include a polyamide polymer. In some cases, the outer layer may include a polyimide polymer.

In some cases, the plurality of electrodes may be arranged in an x by y grid, where x and y are each integers that are equal to or greater than one. As an example, x may range from one to ten and y may range from one to ten. In some cases, each of the plurality of electrodes may be electrically isolated from each other. In some cases, each of the plurality of electrodes may be individually actuatable by electrically coupling a source of RF (radiofrequency) energy with individual electrodes of the plurality of electrodes. In some cases, the electrode assembly may be adapted to allow varying lacerating or cauterizing patterns by individually actuating individual electrodes of the plurality of electrodes. In some cases, the electrode assembly may include two or more axially aligned electrodes. In some cases, the electrode assembly may include two or more elongate electrodes that are not parallel to each other. In some cases, the plurality of electrodes may include one or more monopolar electrodes.

In some instances, a medical device is adapted for lacerating cardiac tissue. The medical device includes an elongate shaft that extends proximally from a distal region and an inflatable balloon that is secured to the distal region. The inflatable balloon has an outer surface and includes a deflated configuration and an inflated configuration. An electrode assembly is disposed on the outer surface. The electrode assembly includes an insulative surface that is adhered to the outer surface and a conductive pattern that is formed on the insulative surface. The conductive pattern defines a plurality of electrodes.

In some cases, the inflatable balloon may include a polyimide polymer or a polyamide polymer. In some cases, each of the plurality of electrodes may be electrically isolated from each other and may be individually actuatable. In some cases, the electrode assembly may be adapted to allow varying cutting patterns by individually actuating individual electrodes of the plurality of electrodes. In some cases, the plurality of electrodes may include two or more axially aligned electrodes. In some cases, the plurality of electrodes may include two or more non-parallel elongate electrodes. In some cases, the electrode assembly may be radiopaque.

In some instances, a medical device is adapted for lacerating and/or cauterizing valve leaflets. The medical device includes an elongate shaft that extends proximally from a distal region and an inflatable balloon that is secured to the distal region. The inflatable balloon includes a polyimide polymer or a polyamide polymer, and has an outer surface. An electrode assembly is disposed relative to the outer surface. The electrode assembly includes a plurality of electrodes that are arranged in an x by y grid, where x and y are each integers that are equal to or greater than one. In some cases, the electrode assembly may be adapted to allow varying cutting patterns by individually actuating individual electrodes of the plurality of electrodes.

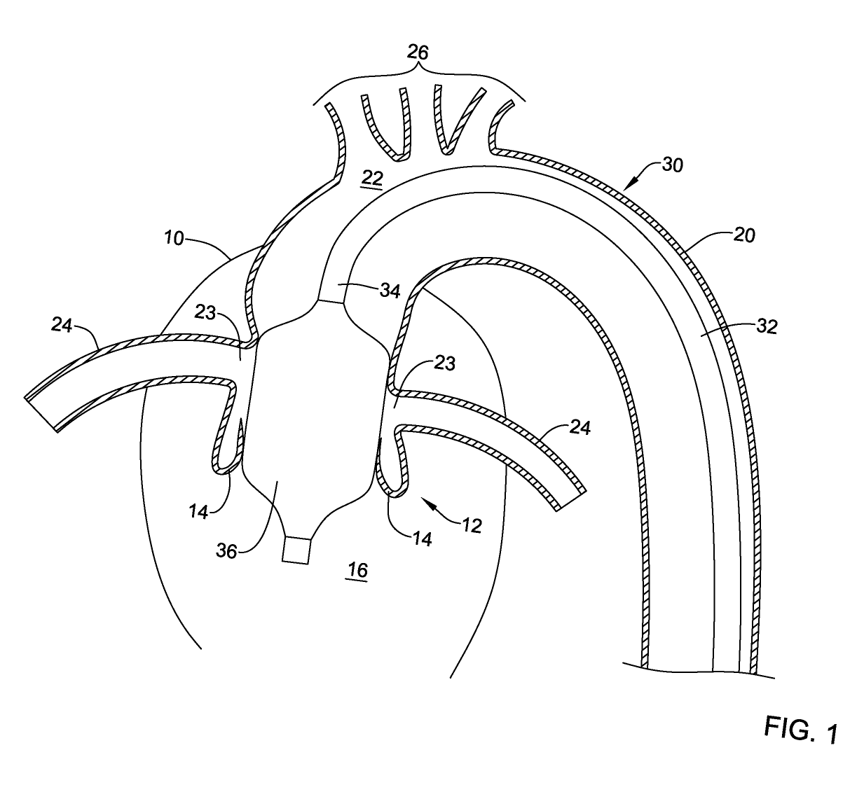

FIG. 1 is a schematic partial cut-away view of a portion of a patient’s heart 10 including an aortic valve 12 having native valve leaflets 14 disposed within and/or extending from a native valve annulus, a left ventricle 16, and certain connected vasculature, such as an aorta 20 connected to the aortic valve 12 of the patient’s heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. While the aortic valve 12 includes a total of three native valve leaflets 14, only two are visible in the illustrated cutaway view. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

As shown in FIG. 1, a medical device 30 has been advanced through the aorta 20 and through the aortic arch 22 to a position in which the medical device 30 extends through the native valve leaflets 14. The medical device 30 includes an elongate shaft 32 that extends proximally from a distal region 34. An inflatable balloon 36 is secured to the distal region 34 and is movable between a deflated configuration and an inflated configuration (as shown). While the medical device 30 is shown compressing the native valve leaflets 14, it will be appreciated that the medical device 30 may also be used to compress and/or lacerate artificial valve leaflets in a previously implanted replacement heart valve before implanting a new replacement heart valve within the previously implanted replacement heart valve. Some non-limiting examples of a replacement heart valves with which the medical device 30 may be utilized include the ACURATE NEO2™, the ACURATE PRIME™, and/or family members thereof from Boston Scientific of Marlborough, MA, USA.

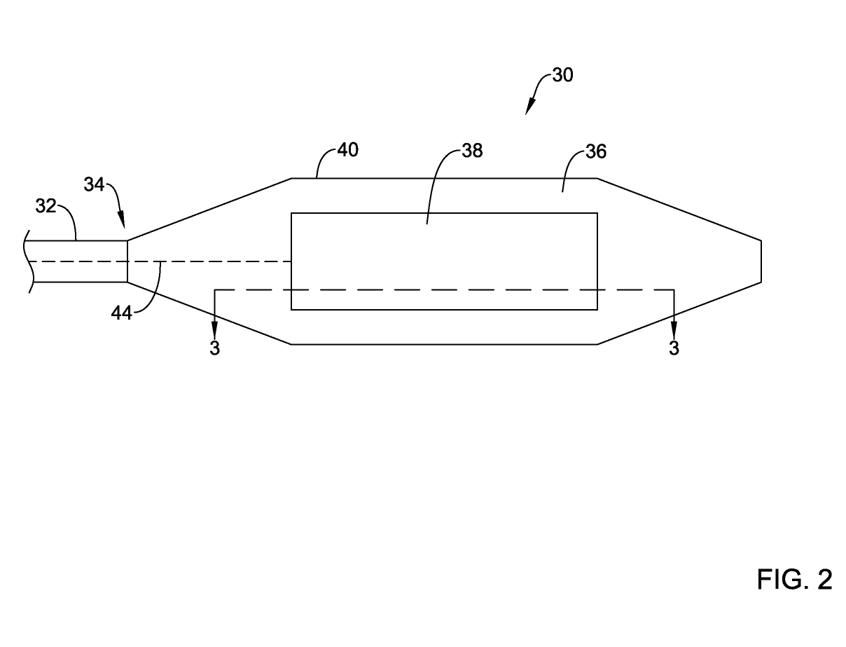

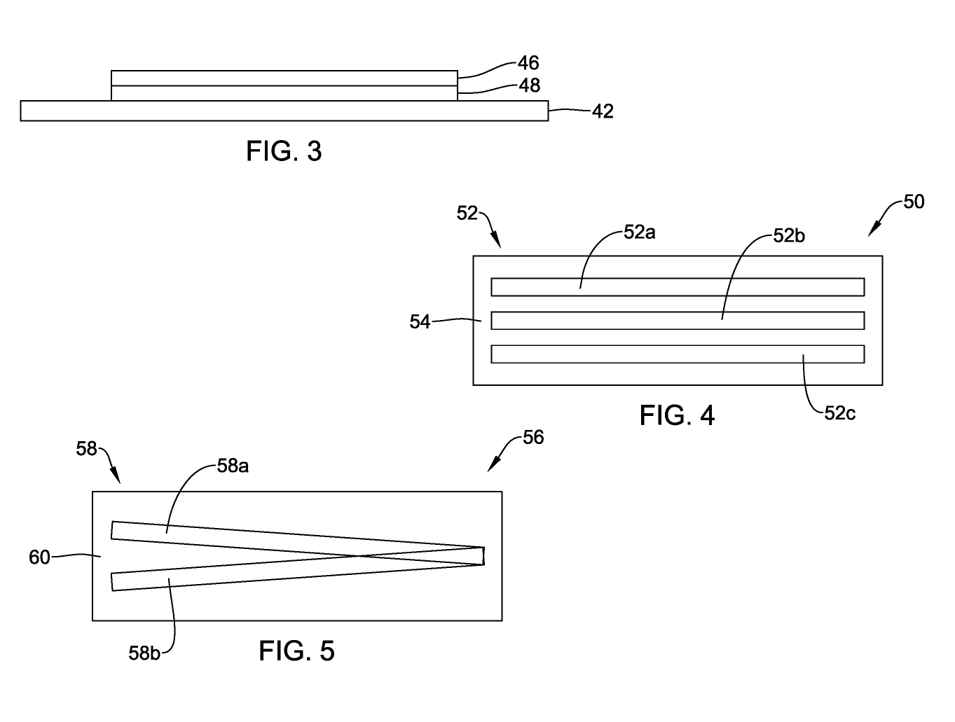

FIG. 2 is a schematic view of the illustrative medical device 30. The illustrative medical device 30 includes an electrode assembly 38 that is disposed on an outer surface 40 of the inflatable balloon 36. As seen for example in FIG. 3, which is a cross-sectional view taken along the line 3-3 of FIG. 2, the inflatable balloon 36 may be considered as having a balloon wall 42 that forms an outermost layer of the inflatable balloon 36. The balloon wall 42 may form the outer surface 40, for example. In some cases, the balloon wall 42 may be a single polymeric layer. In some cases, the balloon wall 42 may represent two or more distinct polymeric layers that form the balloon wall 42. In some cases, the inflatable balloon 36 may include a polyimide polymer or a polyamide polymer. In some cases, the balloon wall 42 may represent two or more layers. As an example, an inner layer may be a polyamide polymer and an outer layer may be a polyimide polymer.

The electrode assembly 38 may be electrically coupled with an electrical conductor 44. While shown as a single line, the electrical conductor 44 may represent two or more electrical conductors that are adjacent each other or coaxial and that are coupled to each of a plurality of electrodes forming part of the electrode assembly 38, as will be discussed. With reference to FIG. 3, the electrode assembly 38 may include an upper conductive layer 46 and a lower insulative layer 48. The conductive layer 46 may be formed of an electrically conducting medium, such as copper, silver, platinum iridum, or a conductive polymer such as polyimide. The conductive layer 46 may be printed, deposited, molded, or inserted into the insulative layer 48. The conductive layer 46 may include foiled layers, or wires, for example. In some cases, the conductive layer 46 may define not only the electrodes within the electrode assembly 38, but also the conductive traces that electrically connect to each of the electrodes within the electrode assembly 38. The insulative layer 48 may be formed of any insulative polymer that is able to be adhered to the outer surface 40.

FIG. 4 is a schematic view of an electrode assembly 50 that may be used in place of the electrode assembly 38 as part of the medical device 30. The electrode assembly 50 includes several electrodes 52, individually labeled as 52a, 52b, and 52c, disposed on an underlying insulative layer 54. In some cases, as shown, the electrodes 52 may be considered as axially extending electrodes. In some cases, the electrodes 52 may instead be circumferentially extending electrodes. In some cases, the insulative layer 54 may be considered as an example of the insulative layer 48 shown in FIG. 3.

In some cases, as shown, the electrodes 52 may be considered as being parallel with each other. While a total of three electrodes 52 are shown, in some cases there may only be a single electrode 52, or two electrodes 52. In some cases, there may be four or more electrodes 52. In some cases, one or more of the electrodes 52 may be actuated (connected to a source of RF energy) while others of the electrodes 52 are not actuated. In some cases, two or more of the electrodes 52 may be sequentially actuated. In some cases, each of the electrodes 52 may be a single elongate electrode. In some cases, each of the electrodes 52 may be formed from a gridwork of smaller electrodes that can be addressed and actuated together. While a single conductive layer 46 and a single insulative layer 48 are shown, it will be appreciated that this is merely illustrative, as the conductive layer 46 may include two or more conductive layers, and the insulative layer 48 may include two or more insulative layers. In some cases, the insulative layer 48 may be thermally insulating as well as electrically insulating. In some cases, the conductive layer 46 may be embedded within the insulative layer 48.

FIG. 5 is a schematic view of an electrode assembly 56 that may be used in place of the electrode assembly 38 as part of the medical device 30. The electrode assembly 56 includes several elongate electrodes 58, individually labeled 58a and 58b that are disposed on an underlying insulative layer 60. The insulative layer 60 may be considered as being an example of the insulative layer 48 shown in FIG. 3. In some cases, the elongate electrodes 58 are not parallel, but rather intersect each other. The elongate electrodes 58 may provide a cutting pattern having two cuts, forming a flap extending therebetween. In some cases, the elongate electrodes 58 may be actuated simultaneously or sequentially. In some cases, each of the elongate electrodes 58 may be a single elongate electrode. In some cases, each of the elongate electrodes 58 may be formed from a gridwork of smaller electrodes that can be addressed and actuated together.

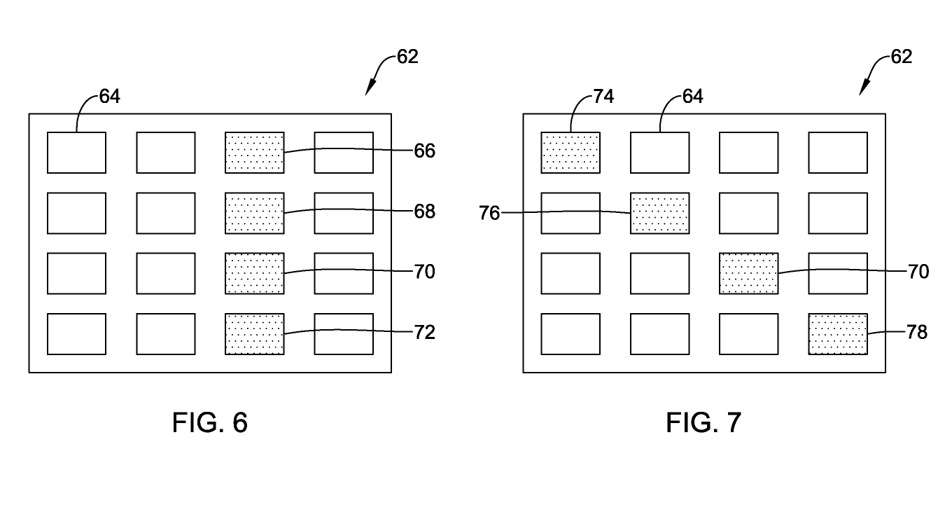

In some cases, an electrode assembly may include a plurality of individual electrodes that can be selectively actuated in order to form a particular cutting pattern. FIGS. 6 through 9 provide example schematic views of an electrode assembly 62 that may be used in place of, or in conjunction with, the electrode assembly 38 as part of the medical device 30. The electrode assembly 62 may be considered as being a grid of individual electrodes 64. In some cases, each of the individual electrodes 64 are mono-polar electrodes, meaning that a grounding pad is placed somewhere on the patient when the electrode assembly 62 is being used. In some cases, at least some of the individual electrodes 64 may be bi-polar, tri-polar, or even quad-polar. In some cases, a particular individual electrode 64 may be used to apply RF energy, and another particular individual electrode 64 that is spaced apart from the first particular individual electrode 64 may function as a ground electrode. A variety of combinations of electrodes 64 may be contemplated.

In some cases, each of the individual electrodes 64 are electrically isolated from each other. In some cases, each of the individual electrodes 64 are individually addressable and actuatable. In some cases, the electrode assembly 62 may be considered as including a number of individual electrodes 64 that are arranged in an x by y grid, with a total number of electrodes 64 equal to x*y. In some cases, x and y may each be integers and may each range from one to ten. As shown, x and y are each equal to four, so there are a total of sixteen electrodes 64 within the electrode assembly 62.

By selectively actuating particular electrodes 64 within the electrode assembly 62, varying cutting patterns may be achieved. FIG. 6 shows a cutting pattern that may result in a cut being made that is transverse to the inflatable balloon 36. In FIG. 6, specific electrodes 66, 68, 70, and 72 have been actuated. The specific electrodes 66, 68, 70, and 72 may be actuated sequentially, randomly, or simultaneously. FIG. 7 shows a cutting pattern that may result in a cut that extends across the inflatable balloon 36 at an acute angle. In FIG. 7, specific electrodes 74, 76, 70, and 78 have been actuated. The specific electrodes 74, 76, 70, and 78 may be actuated sequentially, randomly, or simultaneously. In some cases, the cutting pattern shown in FIG. 7 may correspond to one of the elongate electrodes 58 shown in FIG. 5.

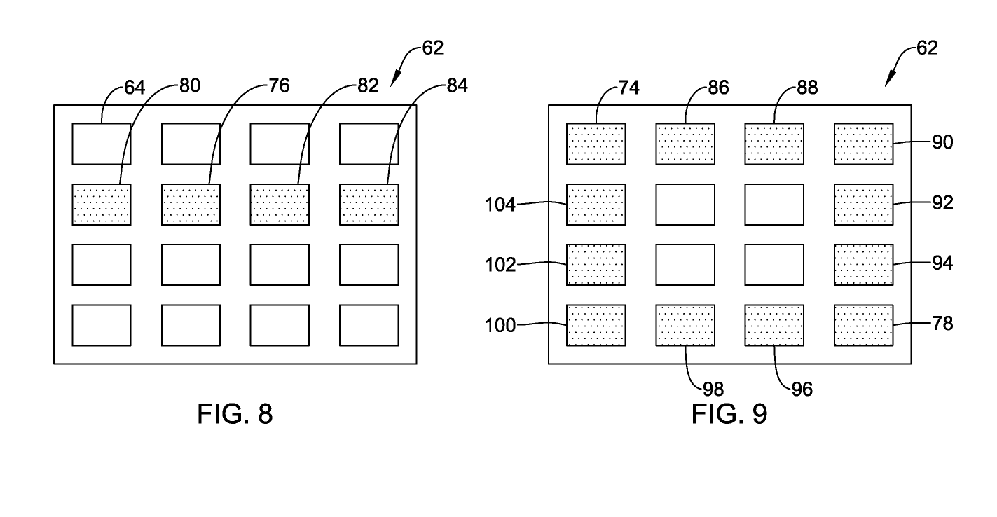

FIG. 8 shows a cutting pattern that may result in a cut that extends axially relative to the inflatable balloon 36. In FIG. 8, specific electrodes 80, 76, 82, and 84 have been actuated. The specific electrodes 80, 76, 82, and 84 may be actuated sequentially, randomly, or simultaneously. In some cases, the cutting pattern shown in FIG. 8 may correspond to one of the axially extending electrodes 52 shown in FIG. 4. FIG. 9 shows a cutting pattern that may result in a cut that roughly forms a circle or rectilinear shape. In FIG. 9, specific electrodes 74, 86, 88, 90, 92, 94, 78, 96, 98, 100, 102, and 104 have been actuated. The specific electrodes 74, 86, 88, 90, 92, 94, 78,96, 98, 100, 102, and 104 may be actuated sequentially, randomly, or simultaneously. This cutting pattern may result in a piece of a valve leaflet 14 to be cut loose from the valve leaflet 14.

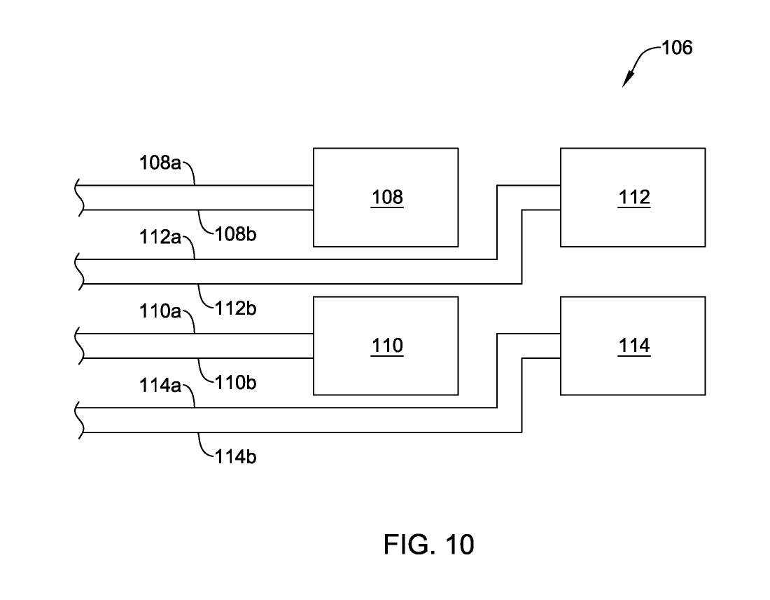

As noted, each of the electrodes 64 may be electrically isolated from each other, and may be individually addressable and actuatable. FIG. 10 is a schematic view of a portion of an electrode assembly 106 that includes an electrode 108, an electrode 110, an electrode 112, and an electrode 114. The electrode 108 is electrically coupled with conductive traces 108a and 108b. The electrode 110 is electrically coupled with conductive traces 110a and 110b. The electrode 112 is electrically coupled with conductive traces 112a and 112b. The electrode 114 is electrically coupled with conductive traces 114a and 114b. Collectively, the conductive traces 108a, 108b, 110a, 110b, 112a, 112b, 114a, and 114b may be considered as being part of the electrical conductor 44 shown in FIG. 2.

Additional materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem or VESTAMID® L21 available from Evonik Industries), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyamide, polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure’s scope is, of course, defined in the language in which the appended claims are expressed.

What is claimed is:

1. A medical device adapted for lacerating valve leaflets, the medical device comprising:
an elongate shaft extending proximally from a distal region;
an inflatable balloon secured to the distal region, the inflatable balloon has an outer surface and includes a deflated configuration and an inflated configuration; and
an electrode assembly disposed relative to the outer surface, the electrode assembly including a plurality of electrodes.

2. The medical device of claim 1, wherein the inflatable balloon comprises a polyimide polymer or a polyamide polymer.

3. The medical device of claim 2, wherein the inflatable balloon comprises an inner layer including a polyamide polymer and an outer layer including a polyimide polymer.

4. The medical device of claim 1, wherein the plurality of electrodes are arranged in an x by y grid, where x and y are each integers that are equal to or greater than one.

5. The medical device of claim 4, wherein x ranges from one to ten and y ranges from one to ten.

6. The medical device of claim 1, wherein each of the plurality of electrodes are electrically isolated from each other.

7. The medical device of claim 1, wherein each of the plurality of electrodes are individually actuatable by electrically coupling a source of RF energy with individual electrodes of the plurality of electrodes.

8. The medical device of claim 5, wherein the electrode assembly is adapted to allow varying lacerating or cauterizing patterns by individually actuating individual electrodes of the plurality of electrodes.

9. The medical device of claim 1, wherein the electrode assembly comprises two or more axially aligned electrodes.

10. The medical device of claim 1, wherein the electrode assembly comprises two or more elongate electrodes that are not parallel to each other.

11. The medical device of claim 1, wherein the plurality of electrodes comprise one or more monopolar electrodes.

12. A medical device adapted for lacerating cardiac tissue, the medical device comprising:
an elongate shaft extending proximally from a distal region;
an inflatable balloon secured to the distal region, the inflatable balloon has an outer surface and includes a deflated configuration and an inflated configuration; and
an electrode assembly disposed on the outer surface, the electrode assembly comprising:
an insulative surface adhered to the outer surface; and
a conductive pattern formed on the insulative surface, the conductive pattern defining a plurality of electrodes.

13. The medical device of claim 12, wherein the inflatable balloon comprises a polyimide polymer or a polyamide polymer.

14. The medical device of claim 12, wherein each of the plurality of electrodes are electrically isolated from each other and individually actuatable.

15. The medical device of claim 12, wherein the electrode assembly is adapted to allow varying cutting patterns by individually actuating individual electrodes of the plurality of electrodes.

16. The medical device of claim 12, wherein the plurality of electrodes include two or more axially aligned electrodes.

17. The medical device of claim 12, wherein the plurality of electrodes include two or more non-parallel elongate electrodes.

18. The medical device of claim 12, wherein the electrode assembly is radiopaque.

19. A medical device adapted for lacerating or cauterizing valve leaflets, the medical device comprising:
an elongate shaft extending proximally from a distal region;
an inflatable balloon secured to the distal region, the inflatable balloon comprising a polyimide or a polyamide, the inflatable balloon has an outer surface; and
an electrode assembly disposed relative to the outer surface, the electrode assembly including a plurality of electrodes that are arranged in an x by y grid, where x and y are each integers that are equal to or greater than one.

20. The medical device of claim 19, wherein the electrode assembly is adapted to allow varying cutting patterns by individually actuating individual electrodes of the plurality of electrodes.
